(19) United States
(12) Patent Application Publication
(10) Pub. No.: US 2011/0077450 A1
(43) Pub. Date: Mar. 31, 2011

(54) MAGNETIC REINFORCING AND REDUCING ACUPUNCTURE METHOD EMPLOYING THE IONIZATION TENDENCY OF METALS

(75) Inventor: Han Jo Yang, Busan (KR)

(73) Assignee: HANJI CO., LTD., Busan (KR)

(21) Appl. No.: 12/936,741

(22) PCT Filed: Apr. 2, 2009
(86) PCT No.: PCT/KR2009/001708

§ 371 Date: Oct. 7, 2010

(30) Foreign Application Priority Data
Apr. 7, 2008 (KR) 10-2008-0032358
Apr. 1, 2009 (KR) 10-2009-0028261

(51) Int. Cl. A61N 2/10

(52) U.S. Cl. 600/12

(57) ABSTRACT

The present invention relates to a magnetic reinforcing and reducing acupuncture method employing the ionization tendency of metals, wherein, in the N pole of a permanent magnet, metal ions having a low ionization tendency are used and a large number of metal ions are impregnated into the body together with the magnetic N pole; and, in the S pole of the permanent magnet, a metal having a high ionization tendency is used so that noxious vapor from within the body escapes by means of the metallic ions with the S pole.

### TECHNICAL FIELD

The present invention relates to a magnetic reinforcing and reducing device using the ionization tendency of metals, and more particularly, to a magnetic reinforcing and reducing method using the ionization tendency of metals, wherein metal ions having a low ionization tendency are used in the N pole of a permanent magnet so that a large number of metal ions are impregnated into the body together with the magnetic N pole, and metal ions having a high ionization tendency are used in the S pole of the permanent magnet so that pathogenic Gi or factor escapes from the inside the body by means of the metal ions together with the magnetic S pole, whereby meridian or acupuncture points blocked by waste matter are pierced by the force of magnetic micro-needles and degenerated functions of internal organs are restored.

### BACKGROUND ART

In general, a magnetic therapy based on a meridian theory of a traditional oriental medicine is a folk therapy in which magnets are attached to illness-affected areas suffering from tingling in limbs and lumbus, myalgia, headache and the like, so that pressure and impact are applied to meridian or acupuncture points to diagnose the functions of organic tissues linked to meridian pathways and acupuncture points to promote circulation of blood with magnetic energy due to a strong magnetic force, thereby treating the affected areas.

In this manner, it is known that lines of a magnetic force emitted from the N poles of magnets used in the folk therapy act on bio-current flowing in the human body and hemoglobin whose main ingredient is iron in blood to promote activity of molecules of iron as a ferromagnetic substance, so that when the magnets are attached onto a blood stagnation area, a tender point felt by a patient when being depressed or an illness-affected area with them contacting the skin, a treatment effect is exhibited within several hours or days, which is called a acupuncture point magnetic therapy.

Such an acupuncture point magnetic therapy currently employs the following methods as a method in which a magnet is attached to acupoints linked to meridian pathways by developing a product in which the ionization tendency property of metal is applied to the magnet to thereby increase a treatment effect: a method in which the N pole of the magnet comes into close contact with the skin similarly to a conventional magnetic therapy, a method in which the N pole of the magnet comes into close contact with Sa-Gwan acupuncture points, Sa-Chong acupuncture points and eight acupuncture points of eight extra meridians or a pain area where muscles and nerves run, and a method applying Saamchimbeop or Saam's Acupuncture Method in which the magnet is attached to Five-Shu acupuncture points in joints of the limbs.

In the meantime, as an example of a technology to activate the functions of organic tissues linked to meridian pathways and acupuncture points using magnets, there is proposed Korean Patent No. 1996-8237 (issued on Jun. 21, 1996) which discloses a back support device with a magnet for treatment of acupuncture points. Such a back support device, however, entails a problem in that the backpack support device having a detachable magnet embedded therein is inserted into clothing to treat acupuncture points, causing inconvenience in daily life and leading to an increase in the manufacturing cost due to complexity of the structure thereof.

In addition, Korean Patent Laid-Open Publication No. 2004-6618 (laid open on Jan. 24, 2994) proposes a magnet needle for acupuncture. But this magnet needle encounters a drawback in that since it merely depends on the strength of the magnet, the meridians or the like blocked by waste matter or illness-affected areas are not sufficiently alleviated.

Further, Korean Utility Model Registration No. 20-314460 (issued on May 28, 2003) proposes a magnetic pad in which magnetic powder obtained by pulverizing a magnetic material is applied on the surface of an adhesive so that the magnetic pad is attached on illness-affected areas suffering from myalgia, contusion, arthritis and the like to activate circulation of blood. Such a magnetic pad, however, has a shortcoming in that since long-term use of the pad is impossible, it is low in economic efficiency.

Therefore, to basically address and solve the above-mentioned problems occurring in the prior art, the present inventor has found that metal ions having a low ionization tendency are used in the N pole of a permanent magnet so that a large number of metal ions are impregnated into the body together with the magnetic N pole and metal ions having a high ionization tendency are used in the S pole of the permanent magnet so that pathogenic Gi or factor escapes from the inside the body by means of the metal ions together with the magnetic S pole, whereby meridian or acupuncture points blocked by waste matter are pierced by the force of magnetic micro-needles and degenerated functions of internal organs are restored, and thus completed the present invention.

### DISCLOSURE OF INVENTION

The present invention has been made in order to solve the above-mentioned problems associated with the prior art, and it is an object of the present invention to provide a novel type of magnetic reinforcing and reducing device using the ionization tendency of metals, wherein, an ionic metal plate having a low ionization tendency is covered on the N pole of a permanent magnet so that a large number of metal ions are impregnated into the body together with the magnetic N pole, and an ionic metal plate having a high ionization tendency is covered on the S pole of the permanent magnet so that pathogenic Gi or factor escapes from the inside the body by means of the metal ions together with the magnetic S pole, whereby meridian or acupuncture points blocked by waste matter can be pierced by the force of magnetic micro-needles and degenerated functions of internal organs can be restored.

Another object of the present invention is to provide a novel type of magnetic reinforcing and reducing device using the ionization tendency of metals in which one magnetic pole of the permanent magnet 1 is in close contact with a bottom of an ionic metal plate, a side of the ionic metal plate extends upwardly from a peripheral edge of the bottom to form an aperture at a top end thereof so as to allow an inner space to be defined by the bottom and the side to accommodate the permanent magnet therein, and a pair of opposed tongues is bently protruded toward the inner space from the top end of the side so as to come into close contact with the other magnetic pole of the permanent magnet.

In particular, yet another object of the present invention is to provide a magnetic reinforcing and reducing device using the ionization tendency of metals, in which a coating layer plated with gold (Au) having a low ionization tendency is included in the N pole of the permanent magnet and a coating layer plated with silver (Au) having a higher ionization tendency than that of gold (Au) is included in the S pole of the permanent magnet so as to simplify the structure of the device so that sanitation is ensured since foreign substance or the like does not adhere to the device, and productivity is improved due to simplification of structure.

To achieve the above objects, according to one aspect, the present invention provides a magnetic reinforcing and reducing device using the ionization tendency of metals, including:

a permanent magnet having an N pole and a S pole;

an ionic metal plate coupled to the permanent magnet so as to cover any one of the N and S poles,

wherein in the case where the metal plate is coupled to the N pole of the permanent magnet, the metal plate is formed of at least one metal selected from the group consisting of gold (Au), platinum (Pt), silver (Ag), copper (Cu) and lead (Pb) having a low ionization tendency, or is formed of a coating layer of the metal, and

wherein in the case where the metal plate is coupled to the S pole of the permanent magnet, the metal plate is formed of an aluminum (Al) metal having a high ionization tendency or is formed of a coating layer of the aluminum (Al) metal.

According to another aspect, the present invention provides a magnetic reinforcing and reducing device using the ionization tendency of metals, including:

a permanent magnet having an N pole or an S pole formed on a surface thereof, which is in close contact with the body surface; and

an ionic metal coating layer formed on the N pole or the S pole by plating an outer surface of the permanent magnet with a metal having an ionization tendency.

In the magnetic reinforcing and reducing device of the present invention, preferably, the ionic metal coating layer includes an inner coating layer and outer coating layer.

In the magnetic reinforcing and reducing device of the present invention, preferably, in the ionic metal coating layer formed on the N pole, the inner coating layer is a nickel (Ni) plating layer and the outer coating layer is a gold (Au) plating layer, and in the ionic metal coating layer formed on the S pole, the inner coating layer is a copper (Cu) plating layer and the outer coating layer is a silver (Ag) plating layer.

### EFFECT OF THE INVENTION

According to the magnetic reinforcing and reducing device using the ionization tendency of metals of the present invention as constructed above, when a metal having a low ionization tendency is applied to the N pole of the permanent magnet and is attached to the body surface region of an acupuncture point, a large number of metal ions are impregnated into the body together with the magnetic N pole. When a metal having a high ionization tendency is applied to the S pole of the permanent magnet employed and is attached to the body surface region of an acupuncture point, or if it is attached to the body surface region with the principal acupoints of the hands and feet paired, the magnetic force of the permanent magnet and the property of metal ions stimulates the eight control acupoints and pathogenic Gi or factor escapes from the inside of the body by means of the metal ions together with the magnetic S pole, whereby acupuncture points blocked by waste matter are pierced by the force of magnetic micro-needles and degenerated functions of internal organs are restored.

### BRIEF DESCRIPTION OF THE DRAWINGS

FIG. 1 is a schematic diagrammatic view for explaining a technical spirit of a magnetic reinforcing and reducing device using the ionization tendency of metals according to the present invention;

FIG. 2 is a perspective view showing a magnetic reinforcing and reducing device using the ionization tendency of metals according to a preferred embodiment of the present invention;

FIG. 3 is a cross-sectional view taken along the line A-A of FIG. 2;

FIG. 4 is an exploded perspective view showing a magnetic reinforcing and reducing device using the ionization tendency of metals according to a preferred embodiment of the present invention;

FIG. 5 is a cross-sectional view taken along the line B-B of FIG. 4;

FIG. 6 is a perspective view for explaining a magnetic reinforcing and reducing device using the ionization tendency of metals according to a preferred embodiment of the present invention;

FIG. 7 is a schematic view showing a state in which the magnetic reinforcing and reducing device using the ionization tendency of metals shown in FIG. 6 is in use;

FIG. 8 is a perspective view showing a magnetic reinforcing and reducing device using the ionization tendency of metals according to another preferred embodiment of the present invention;

FIG. 9 is a cross-sectional view taken along the line C-C of FIG. 8; and

FIG. 10 is a perspective view for explaining a magnetic reinforcing and reducing device using the ionization tendency of metals according to another preferred embodiment of the present invention.

### BEST MODE FOR CARRYING OUT THE INVENTION

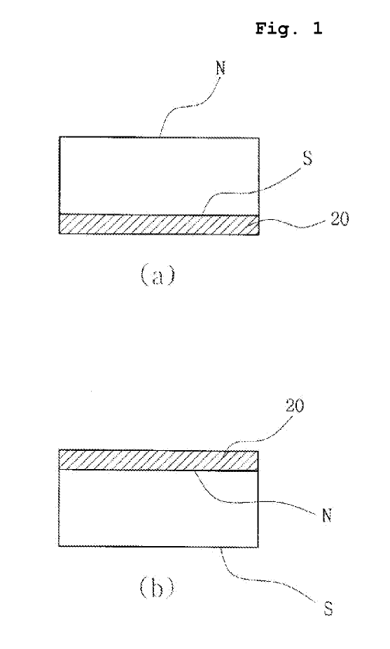

Now, preferred embodiments of the present invention will be described hereinafter in more detail with reference to FIGS. 1 to 10.

In FIGS. 1 to 10, elements performing the same function are denoted by identical reference numerals. In the meantime, in the accompanying drawings, illustration and detailed description of the technical contents of a magnetic reinforcing and reducing method is simplified or omitted, since they can be understood by those skilled in the art, and only portions related with the present invention are shown.

FIG. 1 is a schematic diagrammatic view for explaining a technical spirit of a magnetic reinforcing and reducing device using the ionization tendency of metals according to the present invention.

Referring to FIG. 1, the magnetic reinforcing and reducing device 10 using the ionization tendency of metals according to the present invention includes a permanent magnet 1, and an ionic metal plate or ionic metal coating layer 20.

In case of the permanent magnet 1, it is required that a typical medical magnet which can be attached to the body be thin in thickness, proper in size, and strong in magnetic force. Thus, preferably, a magnet is selectively used which has a magnetic force of from 500 to 3,000 G. When the magnet is attached on the body surface, the N pole may be oriented toward the body surface region of an acupuncture point and the S pole may be oriented toward an opposite side to the body surface region. On the contrary, when the magnet is attached on the body surface, the S pole may be oriented toward the body surface region of an acupuncture point and the N pole may be oriented toward an opposite side to the body surface region.

In addition, the ionic metal plate or the metal coating layer 20 is intended to allow the magnetic force of the magnet to more effectively act on the human body by applying the ionization principle of metals to magnetic force of the magnet. This principle is called an acupuncture point magnetic therapy. In other words, the acupuncture point magnetic therapy is a treatment method in which metal ions having a low ionization tendency are used in the N pole of a permanent magnet so that a large number of metal ions are induced to be impregnated into the body together with the magnetic N pole, and metal ions having a high ionization tendency are used in the S pole of the permanent magnet so that pathogenic Gi or factor is induced to escape from the inside the body by means of the metal ions together with the magnetic S pole, thereby maximizing the treatment effect.

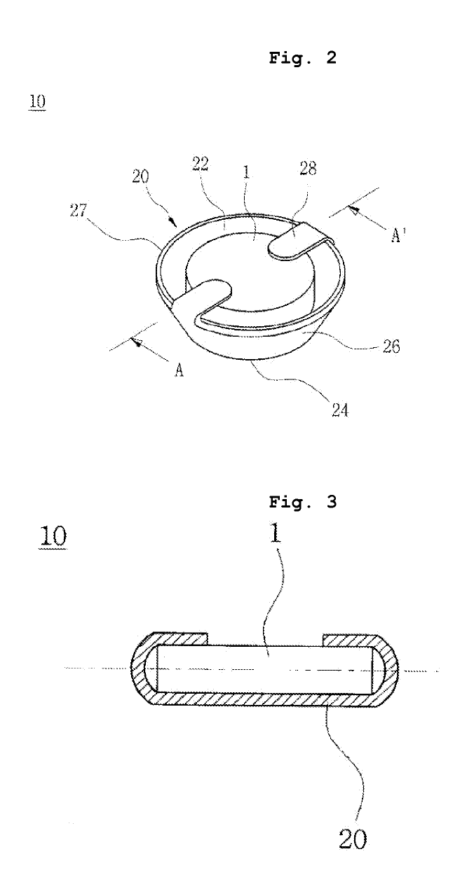

FIG. 2 is a perspective view showing a magnetic reinforcing and reducing device using the ionization tendency of metals according to a preferred embodiment of the present invention, and FIG. 3 is a cross-sectional view taken along the line A-A of FIG. 2.

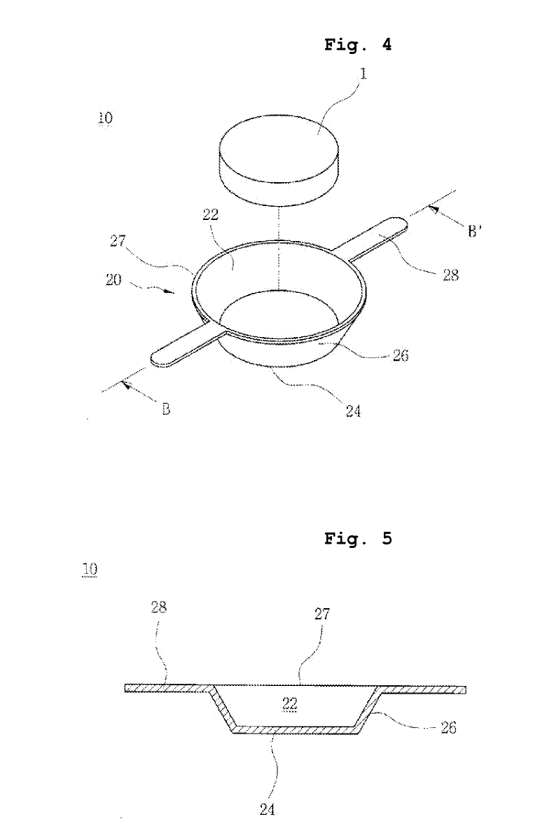

FIG. 4 is an exploded perspective view showing a magnetic reinforcing and reducing device using the ionization tendency of metals according to a preferred embodiment of the present invention, and FIG. 5 is a cross-sectional view taken along the line B-B of FIG. 4.

First, as a preferred embodiment of the present invention, referring to FIGS. 2 and 3, the magnetic reinforcing and reducing device 10 using the ionization tendency of metals according to the present invention includes a permanent magnet 1 and an ionic metal plate 20.

Preferably, the permanent magnet 1 typically uses a magnetic force of from 500 to 3,000 G. When the magnet is attached to the body surface region of an acupuncture point or when it is attached to the body surface region with the principal acupoints of the hands and feet paired, the N pole is preferably oriented toward the acupuncture point or a meridian and the S pole may be oriented toward an opposite side to the body surface region. Alternatively, the N pole and the S pole may be selectively attached to the body surface region of an acupuncture point by a user if the functions of internal organs are degenerated due to waste matter accumulated in the internal organs, if Gi and blood do not flow smoothly due to waste matter accumulated in meridians, or if the flow of GI and blood is interrupted due to waste matter accumulated between cells.

In addition, the ionic metal plate 20 includes: a bottom 24 with which one magnetic pole of the permanent magnet 1 is in close contact; a side 26 extending upwardly from a peripheral edge of the bottom 24 to form an aperture at a top end 27 thereof so as to allow an inner space 22 to be defined by the bottom and the side to accommodate the permanent magnet 1 therein; and a pair of opposed tongues 28 bently protruded toward the inner space from the top end 27 of the side 26 so as to come into close contact with the other magnetic pole of the permanent magnet 1.

An ionic metal constituting the ionic metal plate 20 varies depending on whether a portion contacting the body surface is the N pole or the S pole of the magnet.

That is, a metal suited to the N pole of the magnet is one having a low ionization tendency, which may be at least one metal selected from the group consisting of gold (Au), platinum (Pt), silver (Ag), copper (Cu) and lead (Pb) or a mixture thereof, or may used by being coated using a method such as plating or the like.

A metal suited to the S pole of the magnet is one having a high ionization tendency, which may be an aluminum (Al) metal or may be used by being coated using a method such as plating or the like.

In this manner, the magnetic reinforcing and reducing device 10 using the ionization tendency of metals according to the present invention is constructed such that an ionic metal plate formed of at least one metal selected from the group consisting of gold (Au), platinum (Pt), silver (Ag), copper (Cu) and lead (Pb) having a low ionization tendency or a coating layer of the metal formed by plating or the like is coveringly coupled to the N pole of the permanent magnet 1, and an aluminum (Al) metal plate formed of an aluminum (Al) metal having a high ionization tendency or a coating layer of the metal formed by plating or the like is coveringly coupled to the S pole of the permanent magnet 1. The thus constructed the magnetic reinforcing and reducing device 10 is attached to the body surface region of an acupuncture point or a meridian using a tape or the like with the principal acupoints of the hands and feet paired, if the functions of internal organs are degenerated due to waste matter accumulated in the internal organs, if Gi and blood do not flow smoothly due to waste matter accumulated in meridians, or if the flow of GI and blood is interrupted due to waste matter accumulated between cells. Accordingly, the magnetic reinforcing and reducing device 10 of the present invention has advantage in that the magnetic force of the permanent magnet and the property of metal ions stimulates the eight control acupoints to pierce acupuncture points blocked by waste matter restore degenerated functions of internal organs.

Likewise, the magnetic reinforcing and reducing device using the ionization tendency of metals according to a preferred embodiment of the present invention, as shown in FIGS. 2 and 3, includes an ionic metal plate 20 which includes: a bottom 24 with which one magnetic pole of the permanent magnet 1 is in close contact; a side 26 extending upwardly from a peripheral edge of the bottom 24 to form an aperture at a top end 27 thereof so as to allow an inner space to be defined by the bottom and the side to accommodate the permanent magnet 1 therein; and a pair of opposed tongues 28 bently protruded toward the inner space 22 from the top end 27 of the side 26 so as to come into close contact with the other magnetic pole of the permanent magnet 1. Thus, the permanent magnet 1 is first accommodated in the inner space 22 and then the opposed tongues 28 are bent inwardly of the inner space 22 so that the permanent magnet 1 can be closely fixed to the ionic metal plate 20. Then, the tongues 28 may be properly adjusted in number depending on the demand of consumers or the need of manufacturers.

Meanwhile, referring to FIGS. 4 and 5, the magnetic reinforcing and reducing device 10 using the ionization tendency of metals according to a preferred embodiment of the present invention includes an ionic metal plate 20 which includes: a bottom 24 with which one magnetic pole of the permanent magnet 1 is in close contact; a side 26 extending upwardly from a peripheral edge of the bottom 24 to form an aperture at a top end 27 thereof so as to allow an inner space to be defined by the bottom and the side to accommodate the permanent magnet 1 therein; a pair of opposed tongues 28 extend protrudingly outwardly from the top end 27 of the side 26 as shown in FIG. 4. The permanent magnet 1 is seated in the inner space 22 of the ionic metal plate 20 so that the permanent magnet 1 is surroundingly supported and fixed by the opposed tongues 28 extending protrudingly outwardly from the top end 27 of the side 26

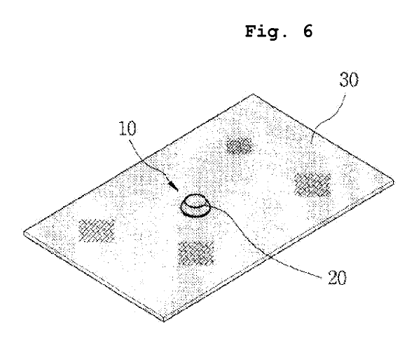

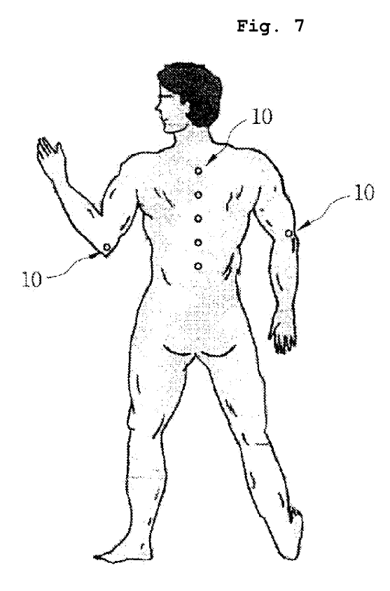

FIG. 6 is a perspective view for explaining a magnetic reinforcing and reducing device using the ionization tendency of metals according to a preferred embodiment of the present invention, and FIG. 7 is a schematic view showing a state in which the magnetic reinforcing and reducing device using the ionization tendency of metals shown in FIG. 6 is in us.

Referring to FIGS. 6 and 7, the magnetic reinforcing and reducing device 10 using the ionization tendency of metals according to a preferred embodiment of the present invention is mounted at the center of the surface of a tape 30 in a state in which the magnet is seated in the inner space of the ionic metal plate 20 and is closely fixed to the ionic metal plate 20 by the opposed tongues (not shown) as shown in FIG. 6. Thus, as shown FIG. 7, if the functions of internal organs are degenerated due to waste matter accumulated in the internal organs, if Gi and blood do not flow smoothly due to waste matter accumulated in meridians, or if the flow of GI and blood is interrupted due to waste matter accumulated between cells, so that tingling in limbs and lumbus, myalgia, headache and the like occur, the magnetic reinforcing and reducing device 10 using the ionization tendency of metals 10 is attached to the body surface regions of meridians and acupuncture points of Saamchimbeop, eight extra meridians, Sa-Gwan acupuncture points and Sa-Chong acupuncture points by a user so as to treat the illness-affected areas.

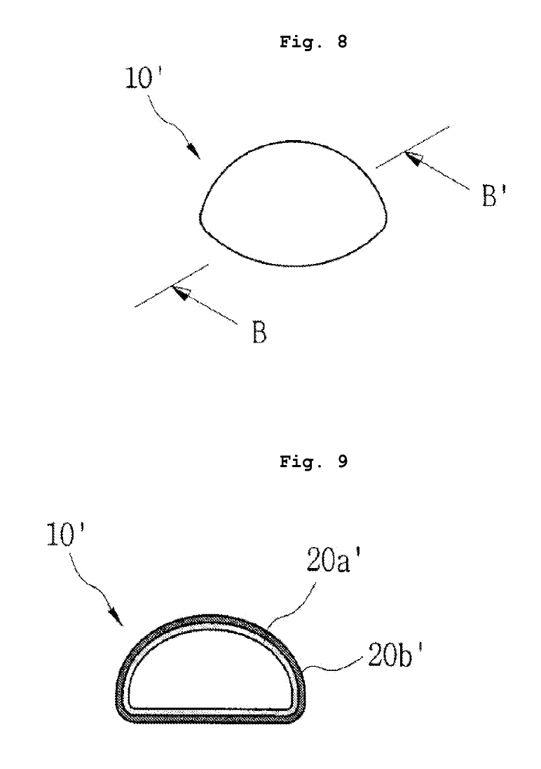

FIG. 8 is a perspective view showing a magnetic reinforcing and reducing device using the ionization tendency of metals according to another preferred embodiment of the present invention, and FIG. 9 is a cross-sectional view taken along the line C-C of FIG. 8.

As another preferred embodiment of the present invention, as shown in FIGS. 8 and 9, the present invention is directed to a magnetic reinforcing and reducing device using the ionization tendency of metals, including: a permanent magnet 1 having an N pole or an S pole formed on a surface thereof, which is in close contact with the body surface; and an ionic metal coating layer 20 formed on the N pole or the S pole by plating an outer surface of the permanent magnet with a metal having an ionization tendency.

In the present invention, the magnetic reinforcing and reducing device in which an N pole or an S pole is formed on a convex surface thereof, contacting with the body surface has the same shape as that of the magnetic reinforcing and reducing device in FIGS. 4 and 5. The magnetic reinforcing and reducing device includes the permanent magnet 1 disposed therein and the ionic metal coating layer 20 having an ionization tendency formed on the permanent magnet, wherein ionic metal coating layer 20 includes an inner coating layer 20a′ for preventing corrosion of the permanent magnet 1 and an outer coating layer 20b′ for protecting the inner coating layer 20a′.

In addition, in the present invention, the surface of the magnetic reinforcing and reducing device, which is in close contact with the body surface is a convex surface opposite to a surface of the tape 30 on which the magnetic reinforcing and reducing device 10′ is attached as shown in FIG. 6.

Moreover, in the present invention, preferably, in the ionic metal coating layer 20 formed on the N pole, the inner coating layer 20a′ is a nickel (Ni) plating layer and the outer coating layer 20b′ is a gold (Au) plating layer, and preferably, in the ionic metal coating layer 20 formed on the S pole, the inner coating layer 20a′ is a copper (Cu) plating layer and the outer coating layer 20b′ is a silver (Ag) plating layer.

In the above construction, the inner coating layer 20a′ of the metal coating layer 20 functions to prevent corrosion of the permanent magnet 1 and increase the bonding force between the permanent magnet 1 and the outer coating layer 20b′. Also, it has been known from ancient times that gold (Au) makes the body warm to act to tonify or reinforce energy of acupuncture points, and silver (Ag) makes the body cold to act to induce pathogenic Gi or factor in the body to acupuncture points to reduce or purge the induced pathogenic Gi or factor. The present invention employs a principle that by the effect of the acupuncture point magnetic therapy is enhanced using the above principle. Thus, the gold (Au) plating layer as the outer coating layer 20b′ formed on the N pole of the magnet is a coating layer formed by being plated using gold (Au) as a metal having a low ionization tendency. Therefore, in order to effectively the magnetic force of the magnet act on the human body by applying the ionization principle of metals to the magnetic force, gold (Au) having a low ionization tendency is used in the N pole of the permanent magnet so that a large number of metal ions are impregnated into the body together with the magnetic N pole. Also, the silver (Ag) plating layer as the outer coating layer 20b′ formed on the S pole of the magnet is a coating layer formed by being plated using silver (Ag) as a metal having a higher ionization tendency than that of gold (Au). Therefore, silver (Au) having a higher ionization tendency is used in the S pole of the permanent magnet so that pathogenic Gi or factor is induced to escape from the inside the body by means of the metal ions together with the magnetic S pole, thereby maximizing the effect of treatment.

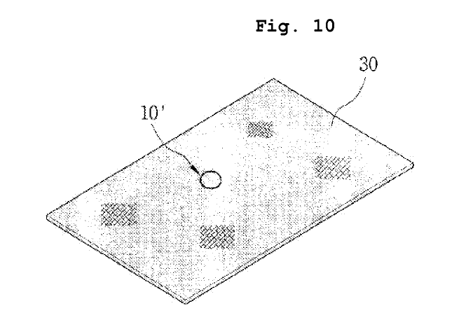

FIG. 10 is a perspective view for explaining a magnetic reinforcing and reducing device using the ionization tendency of metals according to another preferred embodiment of the present invention.

Referring to FIGS. 7 and 10, the magnetic reinforcing and reducing device 10′ using the ionization tendency of metals according to another embodiment of the present invention as shown in FIG. 10.

The magnetic reinforcing and reducing device 10 using the ionization tendency of metals according to a preferred embodiment of the present invention is mounted at the center of the surface of a tape 30 in a state in which an opposed surface to a convex surface in close contact with the body surface is closely fixed to the surface of the tape 30 as shown in FIG. 10. Thus, as shown FIG. 7, if the functions of internal organs are degenerated due to waste matter accumulated in the internal organs, if Gi and blood do not flow smoothly due to waste matter accumulated in meridians, or if the flow of GI and blood is interrupted due to waste matter accumulated between cells, so that tingling in limbs and lumbus, myalgia, headache and the like occur, the convex portion of the magnetic reinforcing and reducing device 10 using the ionization tendency of metals 10 is attached to the body surface regions of meridians and acupuncture points of Saamchimbeop, eight extra meridians, Sa-Gwan acupuncture points and Sa-Chong acupuncture points by a user so as to treat the illness-affected areas.

In the meantime, among methods of attaching the magnetic reinforcing and reducing device to the body surface region of the acupuncture points and the meridians interlinked, a method is widely used which attaches magnetic reinforcing and reducing device to the body surface region using Saamchimbeop and eight extra meridians, which will be described hereinafter.

The Saamchimbeop is a treatment method in which a reinforcement and reduction method is performed on pathogenic syndromes, i.e., deficiency syndrome, excess syndrome, cold syndrome and heat syndrome. In other words, The Saamchimbeop employs the acupuncture point magnetic therapy in which the N pole of the magnet is attached on the body surface region which requires reinforcement or tonification due to weak energy (Gi) of internal organs, and the S pole of the magnet is attached on body surface region which requires reduction or purgation due to rigorous pathogenic Gi or factor and accelerated energy of internal organs.

In the foregoing, the reinforcement or tonification and the reduction or purgation refer to a reinforcing method and a reducing method in acupuncture. The reinforcing method is to excite the regenerated functions of various organs and tissues in the human body to accelerate the functions. The reducing method is to suppress the accelerated functions of various organs and tissues in the human body to recover the functions.

In addition, the reinforcing and reducing method includes point selection-based reinforcement and reduction, hand-based reinforcement and reduction and the like. In particular, if the magnetic reinforcing and reducing device is used such that the N pole is attached to the body surface region in the reinforcing method, and the S pole is attached to the body surface region in the reducing method, stimulus is applied to the illness-affected areas by using the magnetic force and the property of metals, thereby achieving an excellent treatment effect.

Further, a conventional Saamchimbeop has a disadvantage in which since treatment is performed using acupuncture or moxibustion despite the excellent effect, Saamchimbeop is very difficult to use due to limitation in time. On the other hand, in the case where the acupuncture point magnetic therapy is used as in the present invention, since magnetic micro-needles are fixed by the tape or the like, the treatment time can be adjusted so that the treatment can be performed irrespective of time, and a user can do activities naturally even in a state in which the magnetic reinforcing and reducing device is attached on the body surface region of the user.

The eight extra meridians are meridian channels to which the magnetic reinforcing and reducing device 10 is attached with the principal acupoints of the hands and feet paired. A method of attaching the magnetic reinforcing and reducing device is conducted in such a fashion that it is attached to acupuncture points of the hands and feet paired by the same number in the order of numbers 1 to 4 in Table 1 below.

TABLE 1

| | 1 | 2 | 3 | 4 |
|---|---|---|---|---|
| Hand | Hugye | Oegwan | Yeolgyeol | Naegwan (Eum |
| Acupuncture | (Governor | (Yang Link | (Conception | Link |
| Points | Channel) | Channel) | Channel) | Channel) |
| Foot | Sinmaek; | Imeup (Belt | Johae (Eum | Gongson |
| Acupuncture | (Yang Heel | Channel) | Heel | (Penetration |
| Points | Channel) | | Channel) | Channel) |

In the above Table 1, in the paired acupuncture points, an acupuncture point where any syndrome is revealed severely is referred to as a principal acupoint, and an acupuncture point paired together with the principal acupoint is referred to as an auxiliary acupoint. That is, even if any syndrome is revealed, one is a principal acupoint and the other is an auxiliary acupoint in the paired acupuncture points. Likewise, when four acupuncture points distributed respectively in hands and feet are combined in the form of the principal acupoint and the auxiliary acupoint, a total of eight acupuncture points can be paired. If these acupuncture points are properly utilized, it helps communicate meridians blocked by waste matter with each other and recover the vigor of the human body along with elimination of local pain. In this case, preferably, the N pole of the magnet is attached to the principal acupoint and the S pole of the magnet is attached to the auxiliary acupoint.

As described above, in the case where the magnetic reinforcing and reducing device is attached to the body surface regions of acupuncture points and meridians, or is attached to the body surface regions by using the tape or the like with the principal acupoints of the hands and feet paired, eight control acupoints are stimulated by the magnetic force and the property of metal ions so that acupuncture points blocked by waste matter are pierced by the force of the magnetic micro-needles and degenerated functions of internal organs are restored. Furthermore, advantageously, a user can live a daily life without any inconveniences. In the meantime, although the present invention is limited to attachment of the magnetic reinforcing and reducing device using the tape, a method of attaching the magnetic reinforcing and reducing device can be performed in various forms depending on the demand of consumers or the need of manufacturers.

While the magnetic reinforcing and reducing device using the ionization tendency of metals according to the preferred embodiments of the present invention has been described and illustrated in connection with specific exemplary embodiments with reference to the accompanying drawings, it will be readily appreciated by those skilled in the art that it is merely illustrative of the preferred embodiments of the present invention and various modifications and changes can be made thereto within the technical spirit and scope of the present invention.

### MODE FOR INVENTION

To achieve the above objects, according to one aspect, the present invention provides a magnetic reinforcing and reducing device using the ionization tendency of metals, including:

a permanent magnet having an N pole and a S pole;

an ionic metal plate coupled to the permanent magnet so as to cover any one of the N and S poles,

wherein in the case where the metal plate is coupled to the N pole of the permanent magnet, the metal plate is formed of at least one metal selected from the group consisting of gold (Au), platinum (Pt), silver (Ag), copper (Cu) and lead (Pb) having a low ionization tendency, or is formed of a coating layer of the metal, and

wherein in the case where the metal plate is coupled to the S pole of the permanent magnet, the metal plate is formed of an aluminum (Al) metal having a high ionization tendency or is formed of a coating layer of the aluminum (Al) metal.

According to another aspect, the present invention provides a magnetic reinforcing and reducing device using the ionization tendency of metals, including:

a permanent magnet having an N pole or an S pole formed on a surface thereof, which is in close contact with the body surface; and

an ionic metal coating layer formed on the N pole or the S pole by plating an outer surface of the permanent magnet with a metal having an ionization tendency.

In the magnetic reinforcing and reducing device of the present invention, preferably, the ionic metal coating layer includes an inner coating layer and outer coating layer.

In the magnetic reinforcing and reducing device of the present invention, preferably, in the ionic metal coating layer formed on the N pole, the inner coating layer is a nickel (Ni) plating layer and the outer coating layer is a gold (Au) plating layer, and in the ionic metal coating layer formed on the S pole, the inner coating layer is a copper (Cu) plating layer and the outer coating layer is a silver (Ag) plating layer.

### INDUSTRIAL APPLICABILITY

The present invention relates to a magnetic reinforcing and reducing device using the ionization tendency of metals, and more particularly, to a magnetic reinforcing and reducing method using the ionization tendency of metals, wherein metal ions having a low ionization tendency are used in the N pole of a permanent magnet so that a large number of metal ions are impregnated into the body together with the magnetic N pole, and metal ions having a high ionization tendency are used in the S pole of the permanent magnet so that pathogenic Gi or factor escapes from the inside the body by means of the metal ions together with the magnetic S pole, whereby meridian or acupuncture points blocked by waste matter are pierced by the force of magnetic micro-needles and degenerated functions of internal organs are restored.

1. A magnetic reinforcing and reducing device using the ionization tendency of metals, comprising:
a permanent magnet having an N pole and a S pole;
an ionic metal plate coupled to the permanent magnet so as to cover any one of the N and S poles,
wherein in the case where the metal plate is coupled to the N pole of the permanent magnet, the metal plate is formed of at least one metal selected from the group consisting of gold (Au), platinum (Pt), silver (Ag), copper (Cu) and lead (Pb) having a low ionization tendency, or is formed of a coating layer of the metal, and
wherein in the case where the metal plate is coupled to the S pole of the permanent magnet, the metal plate is formed of an aluminum (Al) metal having a high ionization tendency or is formed of a coating layer of the aluminum (Al) metal.

2. The device according to claim 1, wherein the ionic metal plate comprises:
a bottom with which one magnetic pole of the permanent magnet is in close contact;
a side extending upwardly from a peripheral edge of the bottom to form an aperture at a top end thereof so as to allow an inner space to be defined by the bottom and the side; and
a pair of opposed tongues bently protruded toward the inner space from the top end of the side so as to come into close contact with the other magnetic pole of the permanent magnet.

3. A magnetic reinforcing and reducing device using the ionization tendency of metals, comprising:
a permanent magnet having an N pole or an S pole formed on a surface thereof, which is in close contact with the body surface; and
an ionic metal coating layer formed on the N pole or the S pole by plating an outer surface of the permanent magnet with a metal having an ionization tendency.

4. The magnetic reinforcing and reducing device according to claim 3, wherein the ionic metal coating layer comprises an inner coating layer and outer coating layer.

5. The magnetic reinforcing and reducing device according to claim 4, wherein in the ionic metal coating layer formed on the N pole, the inner coating layer is a nickel (Ni) plating layer and the outer coating layer is a gold (Au) plating layer, and in the ionic metal coating layer formed on the S pole, the inner coating layer is a copper (Cu) plating layer and the outer coating layer is a silver (Ag) plating layer.
